(19) United States
(12) Patent Application Publication
(10) Pub. No.: US 2025/0325226 A1
(43) Pub. Date: Oct. 23, 2025

(54) Implant Connectivity

(71) Applicant: Howmedica Osteonics Corp., Mahwah, NJ (US)

(72) Inventors: Andrew Meyer, Davie, FL (US); Matthias Verstraete, Chaam (NL); Chad Bean, Norton, MA (US); Wael Hazin, Plantation, FL (US)

(21) Appl. No.: 18/642,153

(22) Filed: Apr. 22, 2024

(51) Int. Cl. A61B 5/00; A61B 5/07

(52) U.S. Cl. CPC A61B 5/4851; A61B 5/076; A61B 5/686; A61B 5/725; A61B 5/7267; A61B 5/7285; A61B 2560/0209; A61B 2562/0219

(57) ABSTRACT

Disclosed herein is an implant with sensors that can communicate with an external source under predetermined conditions. The implant can include at least one sensor configured to detect implant data. The implant data can be any of an implant condition or adjacent surgical site condition. The implant can include a power source, and a motion switch in communication with the at least one sensor to receive the implant data. A communication module of the implant can be configured to wirelessly communicate with an external source. The motion switch can be configured to initiate wireless communication between the communication module and the external source when the implant data matches a predetermined reference value.

### FIELD OF INVENTION

The present disclosure relates to an implant with sensors, and particularly to an implant with sensors that can communicate with an external source under predetermined conditions.

### BACKGROUND OF THE INVENTION

Implantable medical devices, particularly smart implants—i.e., implants with sensors, encounter challenges in power management due to the constricted confines available for battery integration and the inherent spatial limitations. A smart implant is generally designed to achieve exceptional energy efficiency to prolong battery life, ensuring sustained operation over prolonged durations, which could extend to several years. This requirement becomes even more critical as the smart implant may need to periodically engage in wireless communication with an external source for data transmission and device configuration, amplifying the challenges associated with preserving battery power.

The process of sending complex implant sensor data, especially at high frequencies or over extended distances, may require substantial energy. Moreover, the need for secure data transmission protocols to protect sensitive health information introduces additional layers of complexity and energy demand. These security measures, while crucial for patient privacy and data integrity, further strain the device's limited energy resources. Maintaining a wireless transceiver of the smart implant in an active state for continuous communication can lead to exorbitant energy consumption rates which could dramatically diminishing the smart implant's operational lifespan from the anticipated years to merely days or weeks.

Therefore, there exists a need for implants with sensors capable of efficient communication with an external source.

### BRIEF SUMMARY OF THE INVENTION

Disclosed herein are implants with sensors that can communicate with an external source under predetermined conditions and related methods for such communication.

In accordance with an aspect of the present disclosure, an implant is provided. An implant according to this aspect may include at least one sensor configured to detect implant data. The implant data may be any of an implant condition or adjacent surgical site condition. The implant may include a power source and a motion switch in communication with the at least one sensor to receive the implant data. A communication module of the implant may be configured to wirelessly communicate with an external source. The motion switch may be configured to initiate wireless communication between the communication module and the external source when the implant data matches a predetermined reference value.

Continuing in accordance with this aspect, the at least one sensor may be any of a pH sensor, a temperature sensor, a pressure sensor, a load sensor, an accelerometer, a gyroscope, an IMU and a Hall sensor operatively coupled to a controller of the implant. The controller may be a microcontroller in communication with the motion switch and the communication module.

Continuing in accordance with this aspect, the implant condition may include any of an implant position, implant orientation, and implant movement. The implant movement may include any of an implant acceleration, velocity, and rotation.

Continuing in accordance with this aspect, the adjacent surgical site condition may include any of a temperature, pressure, and pH.

Continuing in accordance with this aspect, the implant may be a joint implant. The joint implant may be a knee implant including a tibial component and a femoral component. The at least one sensor, the power source, the motion switch, and the communication module may be disposed within the tibial component.

Continuing in accordance with this aspect, the power source may be a battery.

Continuing in accordance with this aspect, the motion switch may include a filter configured to compare the implant data with the reference value.

Continuing in accordance with this aspect, the filter may be a relative motion filter, the implant data may be an implant motion and the reference value may be an implant reference position. The relative motion filter may be configured to compare the implant motion with the implant reference position to initiate the wireless communication between the communication module and the external source when the implant motion matches the implant reference position.

Continuing in accordance with this aspect, the filter may be a position filter. The implant data may be an implant position. The reference value may be a gravity vector. The position filter may be configured to compare the implant position with the gravity vector to initiate the wireless communication between the communication module and the external source when the implant position is at predetermined position relative to the gravity vector.

Continuing in accordance with this aspect, the filter may be a motion sequence filter. The implant data may be an implant motion sequence. The reference value may be an implant reference motion sequence. The motion sequence filter may be configured to compare the implant motion sequence with the implant reference motion sequence to initiate the wireless communication between the communication module and the external source when the implant motion sequence matches the implant reference motion sequence.

Continuing in accordance with this aspect, the filter may be a velocity magnitude filter. The implant data may be an implant velocity. The reference value may be an implant reference velocity. The velocity magnitude filter may be configured to compare the implant velocity with the implant reference velocity to initiate the wireless communication between the communication module and the external source when the implant velocity matches the implant reference velocity.

Continuing in accordance with this aspect, the filter may be a machine learning classifier filter configured to compare the implant data with the reference value to initiate the wireless communication between the communication module and the external source when the implant data matches the reference value.

Continuing in accordance with this aspect, the communication module may be a receiver configured to wirelessly transmit the implant data to the external source.

Continuing in accordance with this aspect, the communication module may be a transceiver configured to wirelessly transmit the implant data to the external source and receive external data from the external source. The external data may include an updated predetermined reference value. The transceiver may transmit the updated predetermined reference value to the motion switch.

Continuing in accordance with this aspect, the external source may be any of a smartphone, computer, tablet, and a network.

Continuing in accordance with this aspect, the motion switch may be configured to transition the implant from a low-power mode to a high-power mode when the implant data matches the predetermined reference value. The implant may consume greater energy from the power source in the high-power mode relative to the low-power mode.

### BRIEF DESCRIPTION OF THE DRAWINGS

A more complete appreciation of the subject matter of the present disclosure and the various advantages thereof can be realized by reference to the following detailed description, in which reference is made to the following accompanying drawings:

FIG. 1 is a schematic view of an implant according to an embodiment of the present disclosure;

FIG. 2 is a flowchart showing steps of an implant communication according to an embodiment of the present disclosure;

FIG. 3 is a flowchart showing steps of an implant communication with a kinematic filter according to an embodiment of the present disclosure;

FIG. 4 is a flowchart showing steps of an implant communication with a kinematic filter according to another embodiment of the present disclosure;

FIGS. 5A-5G show schematic views of various positions for implant communication with the kinematic filter of FIG. 4;

FIG. 6 is a flowchart showing steps of an implant communication with a kinematic filter according to another embodiment of the present disclosure;

FIG. 7 is schematic view of implant motion for implant communication with the kinematic filter of FIG. 6;

FIG. 8 is a flowchart showing steps of an implant communication with a kinematic filter according to another embodiment of the present disclosure, and

FIG. 9 is a flowchart showing steps of an implant communication with a kinematic filter according to another embodiment of the present disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features within a different series of numbers (e.g., 100-series, 200-series, etc.). It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

The term “smart implant” means an implant with at least one sensor. The term “joint implant” means a joint implant system comprising two or more implants. It should be understood that the term “implant performance” as used herein includes implant condition and related patient condition such as the condition of tissue and bone around the implant, etc.

As used herein, the terms “implant” and “smart implant” will be used interchangeably and as such, unless otherwise stated, the explicit use of either term is inclusive of the other term. The terms “kinematic filter” and “motion filter” will be used interchangeably and as such, unless otherwise stated, the explicit use of either term is inclusive of the other term. The terms “power” and “energy” will be used interchangeably and as such, unless otherwise stated, the explicit use of either term is inclusive of the other term.

In describing preferred embodiments of the disclosure, reference will be made to directional nomenclature used in describing the human body. It is noted that this nomenclature is used only for convenience and that it is not intended to be limiting with respect to the scope of the present disclosure. As used herein, when referring to bones or other parts of the body, the term “anterior” means toward the front part of the body or the face, and the term “posterior” means toward the back of the body. The term “medial” means toward the midline of the body, and the term “lateral” means away from the midline of the body. The term “superior” means closer to the head, and the term “inferior” means more distant from the head.

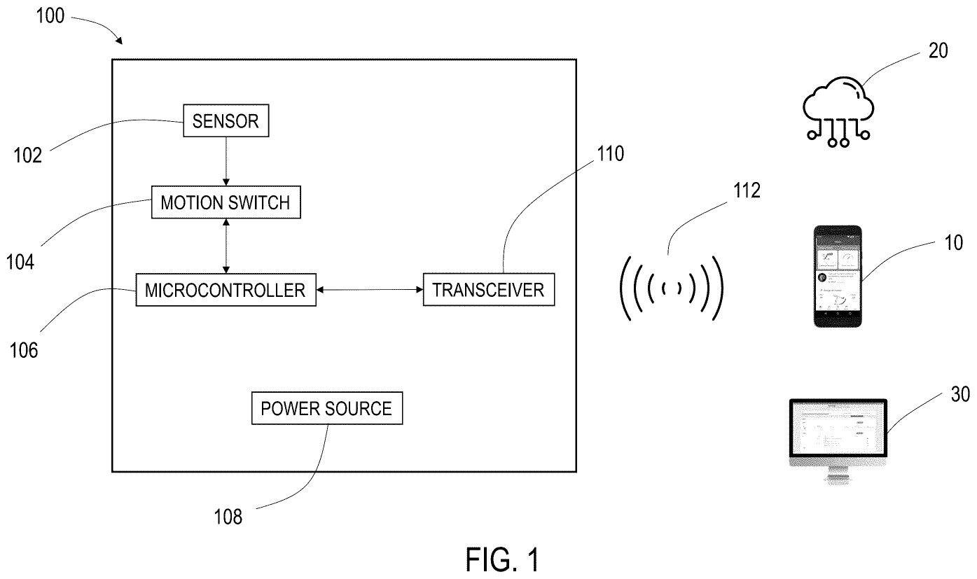

FIG. 1 is a schematic view of an implant 100 according to an embodiment of the present disclosure. Implant 100 includes at least one sensor 102. Sensor 102 can take numerous forms depending on the functional requirements of the implant. Examples of sensor 102 can include, but are not limited to, a load sensor capable of monitoring mechanical stresses, a temperature sensor for detecting thermal variations, a pressure sensor responsive to fluidic or gaseous pressures, an accelerometer for measuring changes in velocity or orientation, a Hall sensor for detecting magnetic fields, a gyroscope for orientation and rotational speed detection, a magnetometer for magnetic intensity measurement, a pH sensor to gauge the acidity or basicity of surrounding tissues or fluids, and an inertial measurement unit (IMU) that integrates multiple sensing capabilities. Sensor 102 can perform a breadth of functions such as measuring, detecting, and comparing various aspects of the implant's performance and its interaction within the surgical site. Taking a knee implant as an example, sensor 102 can be a load sensor specifically designed to accurately measure and record the dynamic loading forces exerted on and by the knee joint during patient activity. Implant 100 can include multiple sensors, each of a different type, to provide a comprehensive monitoring system capable of tracking a range of physiological and biomechanical parameters simultaneously.

Implant 100 includes a power source 108 such as a battery to power the sensor and other electronic components of implant 100. Power source 108 can be an integrated power source 108, such as a rechargeable lithium-ion battery or other suitable energy storage component configured to supply an appropriate and sustained level of electrical energy to various operative elements within implant 100, including but not limited to, sensor 102, control circuitry, and communication apparatus.

A transceiver 110 of implant 100 allows the implant to communicate wirelessly 112 with an external source such as a smartphone 10, cloud 20, computer 30, etc. Transceiver 110 serves a dual purpose within the architecture of implant 100. On one hand, it enables the transmission of collected sensor data and operational status updates from the implant to external devices. On the other hand, it facilitates the reception of incoming instructions or configuration adjustments from an array of external sources. Wireless communication 112 can be realized by the inclusion of a dedicated data transmission element, such as an antenna (not shown). The design and positioning of the antenna within implant 100 are optimized for both the propagation of signals through the human body and minimal interference with surrounding tissues and other medical devices.

Various wireless protocols can be used for the bi-directional communication between implant 100 and the external source including near field communication (“NFC”), Bluetooth Low Energy (BLE), Medical Implant Communication Service (MICS), Wi-Fi, Z-Wave, etc. The selection of a particular wireless protocol for the communication between implant 100 and the external source can be determined by several parameters. One such parameter is the required communication range-different environments and use-cases may dictate different optimal distances for uninterrupted and reliable data transmission. Additionally, the volume and frequency of the data exchange can be considered. Certain clinical applications may necessitate the transfer of large data sets or frequent transmission of sensor readings, thereby requiring a more robust protocol that can manage higher bandwidths and provide greater data throughput. The chosen communication protocol can provide strong encryption and secure connection capabilities to ensure patient confidentiality and integrity of sensitive medical information. Factors such as energy efficiency of the implant's power source, the feasibility of recharging the power source, and the overall power consumption of the communication protocol can influence the protocol selection to preserve the implant's functional longevity and minimize the need for invasive maintenance procedures.

Wirelessly connecting implant 100 to an external source allows the implant to transfer sensor data to the external source and to receive instructions from the external device to change or optimize implant performance if necessary. Ensuring consistent relay of sensor data is contingent upon several factors including patient compliance. This process enables healthcare professionals (HCPs) or surgeons to consistently monitor the status of the implant, such as a knee joint implant, and respond promptly to indicators of malfunction or atypical recovery patterns. However, maintaining such vigilance may prove burdensome for patients. Patients may encounter difficulties with the technological aspects of the monitoring process, such as managing and remembering complex login credentials or navigating the interface of the external device, which can lead to underutilization or neglect. Additionally, powering the implant's sensor array and computational unit demands a battery that not only fits within the spatial constraints of the implant but also has ample energy capacity to ensure sustained functionality. It is imperative for the battery to be durable, stable, and safe within the physiological environment. To facilitate data transfer, implant 100 enters an “advertising mode,” a state where it seeks out and establishes a connection with compatible wireless networks such as Bluetooth Low Energy (BLE). However, being in this mode continuously can lead to accelerated battery depletion, posing a challenge for the energy management system within the implant.

A motion switch 104 of implant 100 as shown in FIG. 1 addresses these concerns by providing a convenient and secure authentication and connectivity between the implant and an external source while simultaneously minimizing battery energy consumption. Motion switch 104 includes a motion filter such as a kinematic filter that conserves power by limiting wireless communications to only occur when specific activities occur. This motion switch is configured to recognize specific movement patterns or kinetic actions associated with the user, which can be utilized as a trigger to initiate bi-directional data transmission between the implant and the external source.

As shown in FIG. 1 and more fully described below motion switch 104 receives sensor data from sensor 102 and initiates bi-directional communication with an external source by activating transceiver 110 via a microcontroller 106. Motion switch 104 is configured to function as a sentinel, receiving a stream of sensor data from sensor 102, which is actively monitoring various parameters such as load, temperature, pressure, or motion within the context of the implant's environment. Upon the acquisition of relevant sensor data, motion switch 104 is programmed to detect predefined movement patterns or thresholds, which, once recognized, trigger the device to pivot from a passive state into an active one, engaging its communication capabilities. Microcontroller 106 can be a specialized and integrated circuit within implant 100 to receive a signal from motion switch 104 and, in response, commands transceiver 110 to initiate bi-directional wireless communication with a designated external source. Thus, transceiver 110 serves as the communicative bridge between implant 100 and the outside world, operates in an optimally timed fashion to exchange vital data between the implant and external devices such as clinicians' monitoring systems, patient interfaces, or medical databases.

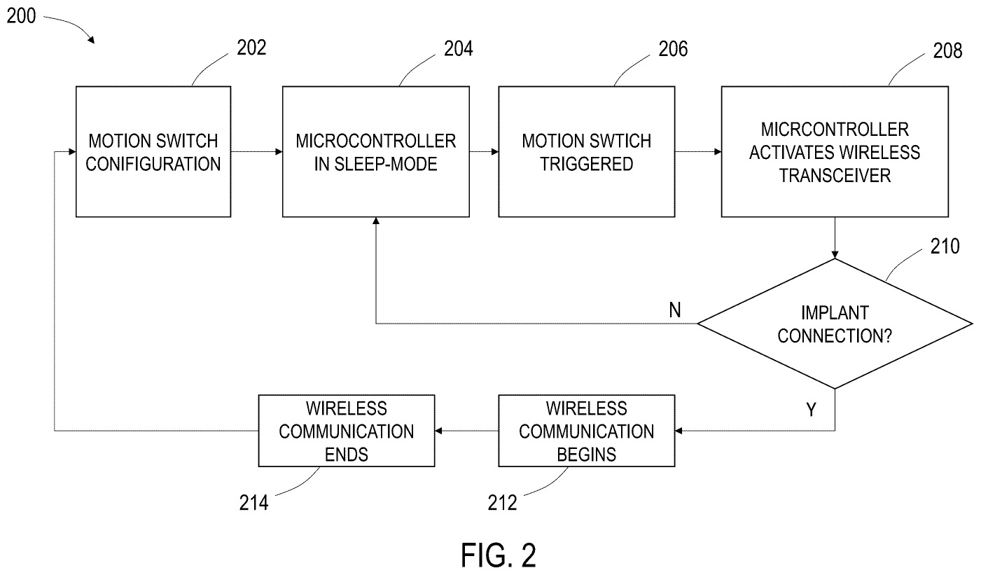

FIG. 2 is a flowchart showing steps of an implant communication 200 with a motion switch according to an embodiment of the present disclosure. These steps are described with reference to the components of implant 100, which is exemplified by a knee joint implant in this specific embodiment. Microcontroller 106 can be used to configure motion switch 104 with specific kinematic filters in a setup step 202. These filters can be calibrated with the user's physiological and biomechanical variables. An example of such customization could involve configuring the kinematic filter to discern a distinct leg shank angle relative to the gravitational vector or a certain relative shank angle motion. This kinematic characteristic, once defined, acts as a reference value, range, threshold, or motion pattern unique to the patient.

In a step 204, microcontroller 106 transitions into a “sleep mode”, a state engineered to significantly reduce the implant's energy consumption when motion switch 104 remains vigilantly operational, albeit operating on a minimal current draw to further preserve energy. When the patient performs an action that matches the predefined kinematic characteristic, motion switch 104 activates microcontroller 106 in a step 206. This action transitions microcontroller 106 from its energy-saving slumber and consequently signals transceiver 110 to awaken and ready itself for data communication processes. Transceiver 110 is poised for activation for a predetermined duration, sufficient to secure a connection with an external source in a step. If this anticipated connection is not achieved within the designated timeframe, microcontroller 106 reverts to its energy-conserving sleep mode in a step 210, thereby conserving implant battery. Contrastingly, upon successful establishment of a connection, a bi-directional communication channel between implant 100 and the external source is established for wireless communication in a step 212. Data transmitted from implant 100 can include information such as sensor data, sensor analytics and other implant metrics, while incoming data from the external source can include operational directives, potentially encompassing sensor calibration refinements, adjustments in sensor operational durations, recommendations on sensor data storage levels, and so forth. Moreover, the calibration parameters of the kinematic filter affiliated with motion switch 104 can be dynamically updated, infusing it with new reference metrics, dynamic ranges, and movement patterns, consequently enhancing the responsiveness and efficacy of the implant's interaction with its user and the environment.

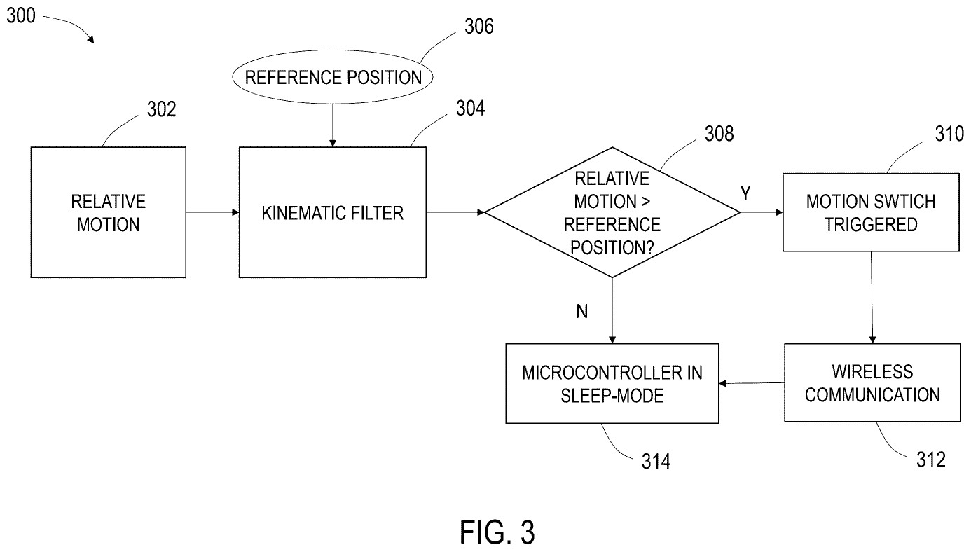

Referring now to FIG. 3, there is shown a flowchart showing steps of an implant communication 300 with a kinematic filter 304 according to an embodiment of the present disclosure. Kinematic filter 304 operates as a relative motion filter to monitor deviations in sensor readings from a pre-established reference position 306 in order to prompt communication data transfer. This relative motion is interpreted as the difference between the sensor's current value and reference position 306, where predetermined deviations initiate the transmission of communication from implant 100 to the external source as more fully described with reference to FIG. 2. The methodology for setting and updating reference position 306 can be defined at the initialization of implant 100, such as during power-up, in one embodiment. Alternatively, the reference position may be recalibrated or reset once the completion of a communication transmission occurs, thus serving as a new benchmark for future motion detection. Additionally, reference position 306 can be programmed to automatically re-establish itself at regular intervals, for instance, every second, creating a dynamic reference that requires sensor deviations to arise within the predefined window to activate communication.

Reference position 306 can take on various forms such as a reference angle or other mathematical constructs that measure orientation. For example, reference position 306 can be represented by a gravitational acceleration vector, wherein a specified magnitude of deviation from this vector (step 308) would trigger the communication process (step 310). The trigger could be an overall vector change or a movement along any singular axis. For example, Euler/Tait-Bryan angles, quaternions, or a direction cosine matrix, which effectively convey the orientation of the implant in three-dimensional space, can be used. In other embodiments, modified Rodriguez parameters or other advanced mathematical descriptors to accurately capture complex relative motions or orientations can serve as reference position 306.

A temporal element can be employed to enhance the specificity of the trigger mechanism. A predetermined delay can be instituted so that only persistent deviations from reference position 306, for example, a delay lasting for a duration of 5 second, would trigger the bi-directional communication. An example of this can involve a knee implant with an IMU, which upon cessation of a prior wireless communication session, records a gravitational vector of [0,1,0] gs, which is thereafter locked in as reference position 306. If the implant is programmed with a threshold for deviation set at 0.25 g, a change in the patient's joint angles that results in a new sensor reading of [0.707,0.707,0] gs would constitute a sufficient deviation, exceeding the established threshold from the reference position, and consequently, trigger the bi-directional communication.

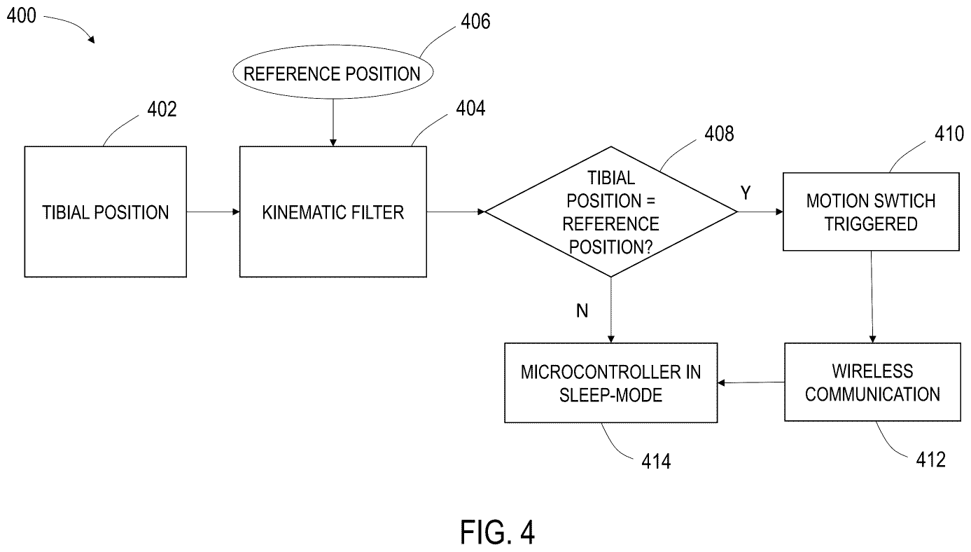

FIG. 4 is a flowchart showing steps of an implant communication 400 with a kinematic filter 404 according to another embodiment of the present disclosure. Implant communication 400 is similar to implant communication 300, and therefore like steps are referred to with similar numerals within the 400-series. However, kinematic filter 404 in this embodiment is an absolute position filter which continuously measures the magnitude and direction of the Earth's gravitational field relative to a predefined vector which serves as a reference position 406. The position of bones in the leg, like the tibia, changes when perform daily activities such as sitting, standing, cycling, lying down, and sleeping. These movements can align with the direction of gravity. Implant communication 400 ensures that implant 100 sends and receives signals only when the leg bone is in a specific position related to gravity.

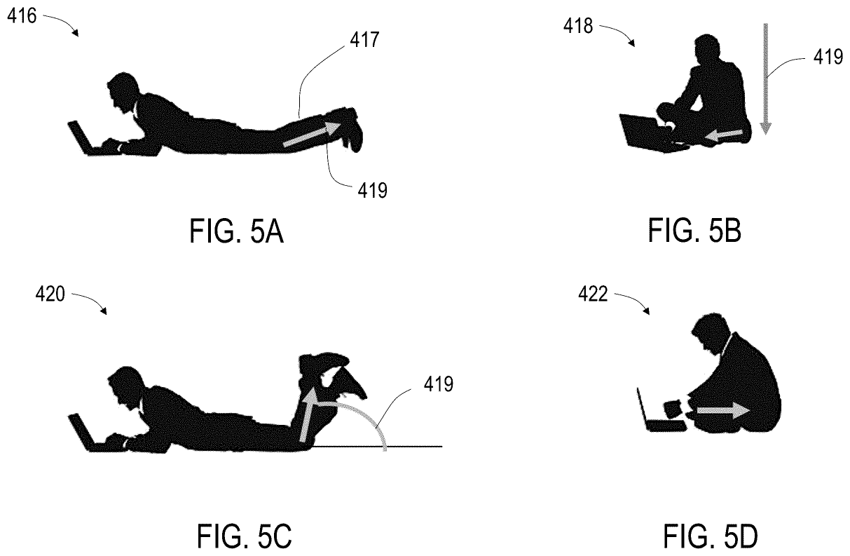

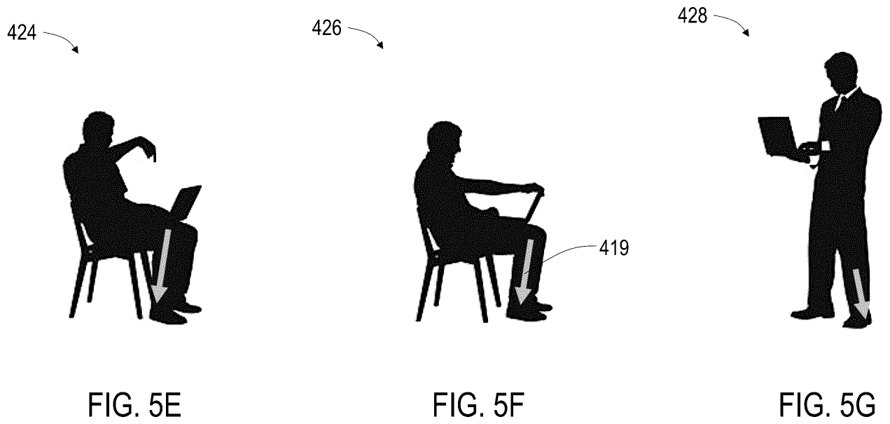

The predefined vector can be parallel to a bone such as the femur, tibia, humerus, etc. For example, FIGS. 5A-5E show a kinematic filter which continuously measures the magnitude and direction of the Earth's gravitational field 421 relative to a predefined vector 419. Vector 419, for the purposes of this embodiment, is geometrically calibrated to maintain alignment with the longitudinal axis of a patient's tibia bone 417. As shown in FIG. 5A, when the patient assumes a recumbent position, causing them to lie down on their back (indicated by 416), vector 419 is generally orthogonal to the gravitational field 421. Similarly, FIGS. 5B and 5D represent scenarios wherein the patient is seated on the ground with legs crossed (denoted by 418) or is in a genuflecting position (illustrated by 422); in both these instances, the vector 419 maintains a perpendicularity to the gravitational field 421 yet is oriented oppositely to the supine posture indicated in 416. FIGS. 5C and 5E-5G portray vector 419 in a generally collinear with the gravitational field 421 when the patient transitions into various seating postures-lying down on their stomach as indicated by 420, sitting down in chairs denoted by 424 and 426, and in an upright stance shown by 428. The alignment of the tibial position, i.e., whether vector 419 coincides with the reference position 406, is monitored. When such an alignment is achieved, motion switch 104 is actuated, thereupon instigating the bi-directional wireless communication as more fully described above with reference to FIG. 2. Thus, implant communication 400 allows for the seamless interplay between the kinematic filter's real-time positional sensing and the subsequent triggering of essential communication protocols for the operation of implant 100.

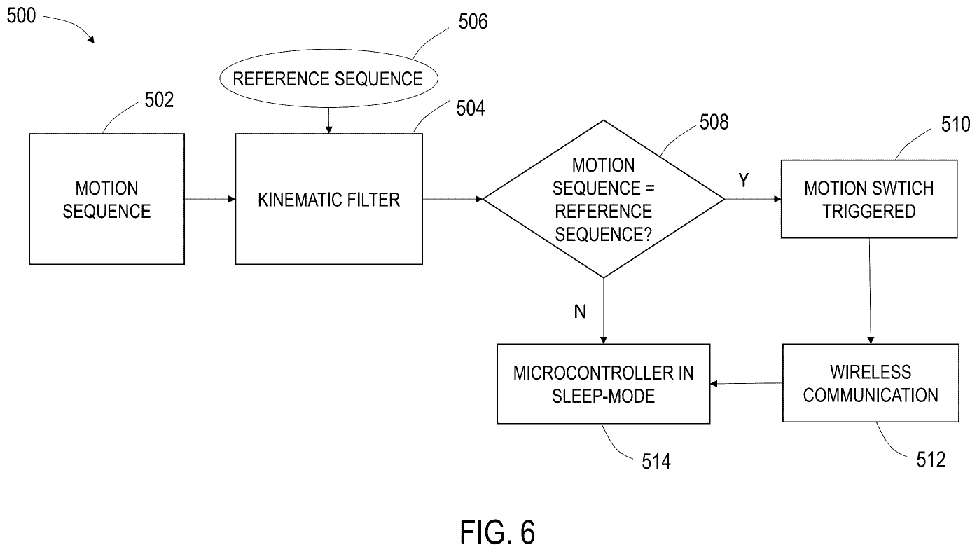

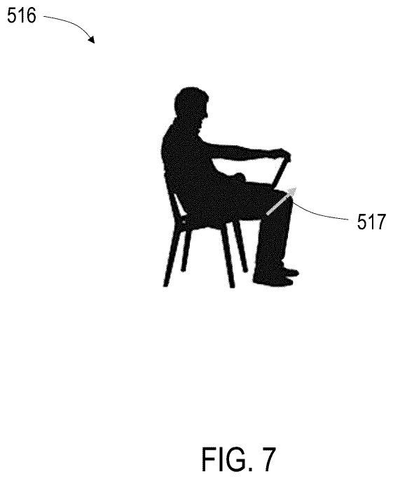

FIG. 6 is a flowchart showing steps of an implant communication 500 with a kinematic filter 504 according to another embodiment of the present disclosure. Implant communication 500 is similar to implant communication 300, and therefore like steps are referred to with similar numerals within the 500-series. However, kinematic filter 504 in this embodiment is configured to detect user inputs, such as a single and/or double physical tap, executed in a specific orientation associated with the user's body posture. An example of this is when the user is in a seated position 516 and conveys the input through a double tap 517 directed onto the knee, oriented medially or laterally as shown in FIG. 7. This gestural interaction serves as input motion sequence 502 detected by sensor 102. If the input motion sequence matches a reference sequence 506 in a step 508, implant 100 activates transceiver 110 to establish bi-directional communication with the external source as described above with reference to FIG. 2.

The enforced specificity of the user input such as double physical tap, its magnitude and phase, is calibrated to be detected by kinematic filter 504. This calibration can be adjusted in order to obviate or substantially diminish any likelihood of false positive readings, wherein non-relevant motions or environmental disturbances might erroneously activate the transceiver. Similarly, the sensitivity and discernment of the filter are honed to reduce or eliminate any incidence of false negatives, where the omission of a legitimate and correctly performed tapping input would otherwise result in a missed activation. Additionally, a precedent condition can be integrated into implant communication 500, such as mandating a predetermined duration of inactivity prior to the tapping input to further bolster the integrity and fault tolerance of kinematic filter 504. This additional criterion of inactivity can serve as a pre-qualification step, augmenting the distinctiveness of the tapping gesture and heightening the overall robustness of the filter's detection mechanism.

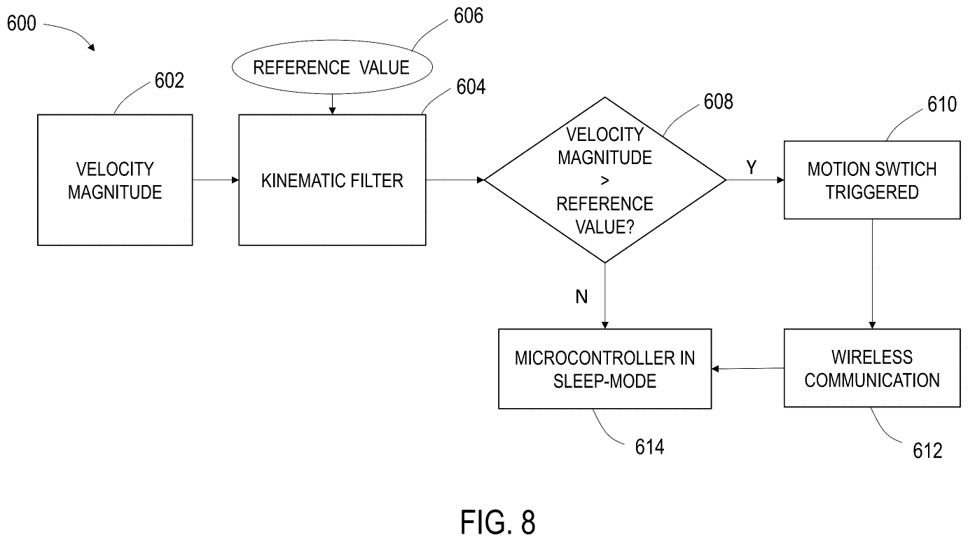

Referring now to FIG. 8, there is shown a flowchart showing steps of an implant communication 600 with a kinematic filter 604 according to another embodiment of the present disclosure. Implant communication 600 is similar to implant communication 300, and therefore like steps are referred to with similar numerals within the 600-series. However, kinematic filter 604 in this embodiment is velocity magnitude filter. An input velocity magnitude 602 detected by sensor 102 is compared with a reference value 606 of kinematic filter 604. If the input velocity magnitude matches reference value 606 in a step 608, implant 100 activates transceiver 110 to establish bi-directional communication with the external source as described above with reference to FIG. 2.

The velocity magnitude filter can be configured to scrutinize the movement of the sensor to transcend mere instantaneous assessments, by instead looking for conditions when the velocity magnitude breaches a particular threshold, which may signify precise states or actions that necessitate implant communication. Velocity can include the rate of change of the gravitational vector components discernible over a specific time frame, or the angular velocity of the sensor, expressible as a vector, quaternion, or through another appropriate angular representation, etc.

To further control the deployment of this filtering algorithm and constrain potential communication overactivities, implant communication 600 can include additional restrictions. For instance, necessitating that the absolute average velocity surpass the threshold for a continuous temporal span (e.g., over 10 seconds), or requiring the velocity to exceed the threshold multiple times to validly prompt communication. It could also place time constraints on these measurements, for example, stipulating that the threshold must be exceeded thrice within a 10-second period.

A parallel approach to triggering implant communication could entail the use of a reference velocity, akin to the positional criteria utilized by kinematic filter 304. This reference velocity threshold may be customized at various instances, subsequent to the cessation of communication, in response to peak velocity occurrence, or based on a timer mechanism.

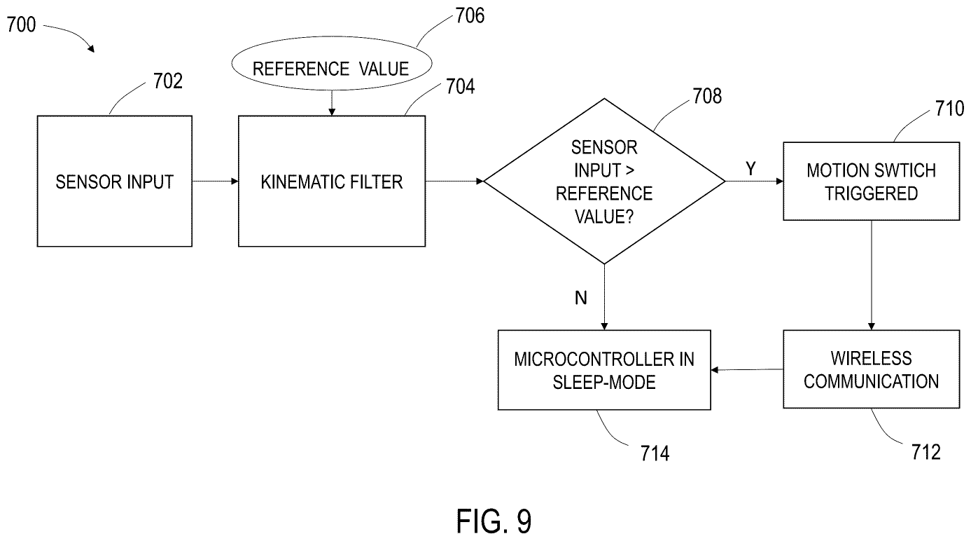

FIG. 9 is a flowchart showing steps of an implant communication 700 with a kinematic filter 704 according to another embodiment of the present disclosure. Implant communication 700 is similar to implant communication 300, and therefore like steps are referred to with similar numerals within the 700-series. However, kinematic filter 704 in this embodiment is machine learning classifier filter. A sensor input 702 detected by sensor 102 is compared with a reference value 706 of kinematic filter 704. If the sensor input matches reference value 706 in a step 708, implant 100 activates transceiver 110 to establish bi-directional communication with the external as described above with reference to FIG. 2.

Machine learning classifier filter can extend beyond typical threshold mechanisms, leveraging algorithms that encompass decision trees, random forest models, support vector machines, or similar machine learning constructs. These algorithms are designed to discern specific activities or motions by analyzing one or multiple sensor inputs.

To facilitate the process of acquiring accurate detection capacities, the machine learning classifier filter undergoes a comprehensive training regimen that is founded upon datasets representative of the implant performing a variety of designated movements or activities. This empirical dataset can be derived from real-life scenarios experienced by individuals with the actual implanted devices or by simulations. Simulated quantitative data, generated through computational means to mimic a person's motion undertaking said activity, can also serve as training fodder for the classifier.

The transformation and preparation of sensor inputs before being received by the machine learning classifier is conducted through various methodologies. This can involve computing metrics across moving time windows, encompassing statistical parameters such as mean, maximum, and minimum values. Sensor readings might also be subjected to mathematical operations such as logarithmic transformations, square-root extractions, or polynomial adjustments, etc., for better characterization. Moreover, sensor data may be differentially or integrally manipulated to highlight dynamic characteristics relevant to the activity being monitored.

Similar to kinematic filter 304, kinematic filter 704 can also refine input data by deducting a reference value benchmarked from the termination point of the most recent communication session, or it may be periodically refreshed to correlate with the ongoing passage of time.

It should be understood that the implants disclosed herein are not limited to a singular mode of communication with an external device. Implant 100 can be configured to incorporate multifaceted communication strategies, either as standalone options or in synergy with the motion switch to enhance the functional connectivity between the implant and external devices for improved performance and user experience. In some embodiments, implant 100 can selectively initiate communication with the external device based on spatial parameters. Correspondingly, the implant can be programmed to recognize and respond to the proximity of an external source. Communication is thus not solely contingent on the motion-triggered signals but can also be activated if the external source is within a pre-established spatial threshold, a predetermined distance from the implant. This geospatial criteria ensure that the implant interacts purposefully with authorized and nearby devices, enhancing security while also conserving system resources.

Moreover, the implants can include a temporal sensitivity in their operational logic. For example, implant 100 can include a temporal monitoring mechanism that observes the attainment of the reference value. In scenarios where this crucial reference value has not been successfully achieved within a designated time frame, such as failing to register over the course of an entire week, the implant can be configured to autonomously activate communication with the appropriate external interface. This fail-safe protocol ensures that, regardless of the specificities surrounding the reference value, critical communication will not be indefinitely deferred. Thus, should the set period surpass its allowed duration, the implant takes intelligent corrective action by initiating communication, thereby maintaining the necessary oversight and management of the device.

While a knee joint implant is disclosed above, all or any of the aspects of the present disclosure can be used with any other implant such as a hip implant, shoulder implant a spinal implant, an intramedullary nail, a bone plate, a bone screw, an external fixation device, an interference screw, etc. Although, the present disclosure generally refers to implants, the systems and method disclosed above can be used with trials to provide real time information related to trial performance. Sensor shape, size and configuration can be customized based on the type of implant and patient-specific needs.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the paragraphs below.

1. An implant comprising:
at least one sensor configured to detect implant data, the implant data being any of an implant condition or adjacent surgical site condition;
a power source;
a motion switch in communication with the at least one sensor to receive the implant data; and
a communication module configured to wirelessly communicate with an external source,
wherein the motion switch is configured to initiate wireless communication between the communication module and the external source when the implant data matches a predetermined reference value.

2. The implant of claim 1, wherein the at least one sensor is any of a pH sensor, a temperature sensor, a pressure sensor, a load sensor, an accelerometer, a gyroscope, an IMU and a Hall sensor operatively coupled to a controller of the implant.

3. The implant of claim 2, wherein the controller is a microcontroller in communication with the motion switch and the communication module.

4. The implant of claim 1, wherein the implant condition include any of an implant position, implant orientation, and implant movement.

5. The implant of claim 4, wherein the implant movement includes any of an implant acceleration, velocity, and rotation.

6. The implant of claim 1, wherein the adjacent surgical site condition includes any of a temperature, pressure, and pH.

7. The implant of claim 1, wherein the implant is a joint implant.

8. The implant of claim 7, wherein the joint implant is a knee implant including a tibial component and a femoral component, the at least one sensor, the power source, the motion switch, and the communication module being disposed within the tibial component.

9. The implant of claim 1, wherein the power source is a battery.

10. The implant of claim 1, wherein the motion switch includes a filter configured to compare the implant data with the reference value.

11. The implant of claim 10, wherein the filter is a relative motion filter, the implant data is an implant motion and the reference value is an implant reference position, the relative motion filter being configured to compare the implant motion with the implant reference position to initiate the wireless communication between the communication module and the external source when the implant motion matches the implant reference position.

12. The implant of claim 10, wherein the filter is a position filter, the implant data is an implant position and the reference value is a gravity vector, the position filter being configured to compare the implant position with the gravity vector to initiate the wireless communication between the communication module and the external source when the implant position is at predetermined position relative to the gravity vector.

13. The implant of claim 10, wherein the filter is a motion sequence filter, the implant data is an implant motion sequence and the reference value is an implant reference motion sequence, the motion sequence filter being configured to compare the implant motion sequence with the implant reference motion sequence to initiate the wireless communication between the communication module and the external source when the implant motion sequence matches the implant reference motion sequence.

14. The implant of claim 10, wherein the filter is a velocity magnitude filter, the implant data is an implant velocity and the reference value is an implant reference velocity, the velocity magnitude filter being configured to compare the implant velocity with the implant reference velocity to initiate the wireless communication between the communication module and the external source when the implant velocity matches the implant reference velocity.

15. The implant of claim 10, wherein the filter is a machine learning classifier filter configured to compare the implant data with the reference value to initiate the wireless communication between the communication module and the external source when the implant data matches the reference value.

16. The implant of claim 1, wherein the communication module is a receiver configured to wirelessly transmit the implant data to the external source.

17. The implant of claim 1, wherein the communication module is a transceiver configured to wirelessly transmit the implant data to the external source and receive external data from the external source.

18. The implant of claim 17, wherein the external data includes an updated predetermined reference value, the transceiver transmitting the updated predetermined reference value to the motion switch.

19. The implant of claim 1, wherein the external source is any of a smartphone, computer, tablet, and a network.

20. The implant of claim 1, wherein the motion switch is configured to transition the implant from a low-power mode to a high-power mode when the implant data matches the predetermined reference value, the implant consuming greater energy from the power source in the high-power mode relative to the low-power mode.
